(19) United States
(12) Patent Application Publication
(10) Pub. No.: US 2010/0112631 A1
(43) Pub. Date: May 6, 2010

(54) Chip Having Microchannel For Counting Specific Micro Particles Among Floating Micro Particle Mixture By Optical Means And A Method For Counting Micro Particles Using The Same

(75) Inventors: Dae Sung Hur, Gyeongsan-si (KR); Chanil Chung, Uiwang-si (KR); Seung Hwa Jeon, Gangneung-si (KR); Jun Keun Chang, Seoul (KR)

Correspondence Address: FENWICK & WEST LLP, SILICON VALLEY CENTER, 801 CALIFORNIA STREET, MOUNTAIN VIEW, CA, 94041, US

(73) Assignee: Digital Bio Technology, Seoul (KR)

(21) Appl. No.: 12/522,377

(22) PCT Filed: Jan. 7, 2008
(86) PCT No.: PCT/KR2008/000071

§ 371 Date: Jul. 7, 2009

(30) Foreign Application Priority Data
Jan. 9, 2007 (KR) 10-2007-0002436

(51) Int. Cl. C12Q 1/06

(52) U.S. Cl. 435/39

(57) ABSTRACT

A microchannel chip for counting floating microparticles with an optical method is provided. The depth of the microchannel chip is made to be a depth of field such that a clear and sharp optical image of microparticles can be obtained, without necessarily diluting the microparticle mixture inside the microchannel. Consequently, a microparticle counting instrument can easily count microparticles shown on an optical image of the microchannel. Particularly, it enables to count more easily the number of CD3 cells CD4 cells or CD8 cells in white blood cells being stained, without lysing or diluting red blood cells. Therefore, the microchannel chip can advantageously used for counting the number of CD3 cells, CD4 cells or CD8 cells in blood of an AIDS patient to monitor the progression of AIDS.

### TECHNICAL FIELD

The present invention relates to a microchannel chip for counting specific microparticles in a microparticle mixture and a method for counting microparticles using the same.

### BACKGROUND ART

One of typical ways to diagnose AIDS, leukemia, or anemia, to monitor how those diseases advance in patients, and to figure out effects of medical treatments is counting the number of leukocytes (white blood cells) or erythrocytes (red blood cells) related to those diseases and examining its distribution in the blood of the patients.

For example, leukocyte has a CD4 cell, a kind of T cell, which is attacked by HIV causing AIDS, used as a breeding ground for further HIV proliferation, and is eventually killed by the virus.

Therefore, an HIV-positive person's CD4 cell count emerges as a useful predictor of current AIDS progression. Suppose that there are 500 or more CD4 cells in 1 ml of blood. This shows the absence of AIDS expression. However, if CD4 cells decline to a range between 200 and 500 in 1 ml of blood, this corresponds to an ARC (AIDS Relate Complex) stage that is previous to the stage of expression of AIDS. However, if 200 or less CD4 cells exist in 1 ml of blood, this means that a person currently has AIDS.

Despite the advantages of CD4 cell counts in blood, sample chips or assay equipment that have been developed for such purpose are very difficult to use and cause many errors.

Therefore, there is a great and growing need to develop a sample chip that can count the number of white blood cells or red blood cells in blood quickly yet accurately and that is easy to use and low in manufacturing cost, and a counting method thereof.

### DISCLOSURE OF INVENTION

### Technical Problem

To solve the above-discussed deficiencies of the related art, a depth of a microchannel in a microchannel chip of the present invention is to be a depth of field such that a clearer optical image of specific microparticles can be captured, without diluting a microparticle mixture in the microchannel. As such, a microparticle counting instrument can easily count microparticles existing in a microchannel, using an optical image of the microchannel.

Therefore, it is an object of the present invention to provide a microchannel chip for optically counting floating microparticles such as white blood cells in blood. It is another object of the present invention to provide a method for counting specific microparticles among a microparticle mixture by using the microchannel chip is provided.

Other objects and advantages of the present invention can be understood by the following description, and become apparent with reference to the embodiments of the present invention. Also, it is obvious to those skilled in the art of the present invention that the objects and advantages of the present invention can be realized by the means as claimed and combinations thereof.

### Technical Solution

In accordance with an aspect of the present invention, there is provided a microchannel chip for counting microparticles. The depth of the microchannel chip of the present invention is a depth of field. The depth of field is determined by magnification of an objective lens, diameter of an iris aperture, a reagent, or a wavelength range, and more details on this will be provided later.

By determining depth of the microchannel in this way, it becomes possible to make microparticles not overlap or aggregate such that a clear and sharp optical image of microparticles may be obtained. This also enables a microparticle counting instrument to count microparticles existing in a microchannel more easily, simply counting microparticles shown on the optical image of the microchannel.

Another aspect of the present invention provides a method for counting microparticles (e.g., white blood cells or CD3, CD4 and CD8 cells) by using the microchannel chip. For an easy count, blood is placed on the microchannel chip and target cells (e.g., white blood cells or CD3, CD4, or CD8 cells) to be counted are stained, without isolation or lysis of red blood cells.

### ADVANTAGEOUS EFFECTS

The microchannel chip of the present invention is fabricated such that the depth of the microchannel is a depth of field. As a result thereof, microparticles are not overlapped or aggregated, and therefore a clear and sharp optical image of microparticles can be obtained. Further, a microparticle counting instrument can easily count microparticles existing in a microchannel simply by counting microparticles shown on an optical image of the microchannel.

Particularly, the present invention enables to count more easily the number of CD3 cells, CD4 cells or CD8 cells in white blood cells being stained, without diluting or lysing red blood cells. Therefore, the present invention can advantageously used for counting the number of CD3 cells, CD4 cells or CD8 cells in blood of an AIDS patient to monitor the progression of AIDS.

### BRIEF DESCRIPTION OF THE DRAWINGS

FIG. 1 and FIG. 2 show respectively a perspective view and a cross-sectional view of a chip having a microchannel.

FIG. 3 is a drawing for explaining a depth of field when observing a sample in a microchannel.

FIG. 4 diagrammatically compares a red blood cell to a white blood cell in blood.

FIG. 5 shows the distribution of white blood cells and red blood cells within the microchannel of the chip according to a preferred embodiment of the present invention.

FIG. 6 and FIG. 7 show CD4 cell observation results, according to one embodiment of the present invention.

### MODE FOR THE INVENTION

The advantages, features and aspects of a microchannel chip and a microparticle counting method using the same will become apparent from the following description of the embodiments with reference to the accompanying drawings, which is set forth hereinafter.

For AIDS diagnosis, a microchannel chip to count CD4 cells in white blood cell is manufactured in this embodiment.

As explained earlier, HIV likes to invade CD4 cells in an AIDS patient and destroy them, and the number of CD4 cells in the patient decreases rapidly. Therefore, by dying CD4 cells and counting the stained CD4 cells, one can diagnose whether a person has developed AIDS and monitor the AIDS progression in a HIV-infected patient.

In general, it is difficult to count only CD4 cell because blood is in a state of suspension containing water, white blood cells with CD4 cells, and red blood cells being dispersed together. Especially, since there are approximately 1000 times more red blood cells, which are easily aggregated, than white blood cells are in human blood, those red blood cells should be lysed, diluted, or isolated from white blood cells, to facilitate CD4 cell counts.

However, according to the present invention, CD4 cells can be stained and counted, without lysing or diluting red blood cells.

For an easy CD4 count, a microchannel chip is manufacturing by obtaining a depth of field and then determining an adequate channel height based on the depth of field.

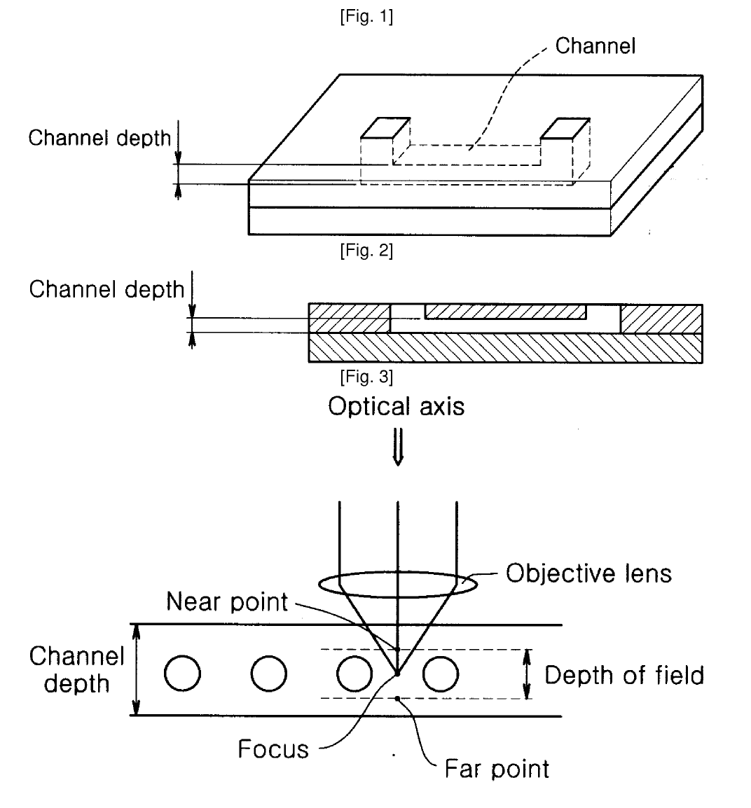

FIG. 1 and FIG. 2 show respectively a perspective view and a cross-sectional view of a microchannel chip. A blood sample (taken from a person) is placed on the microchannel chip, and a microparticle counting instrument takes a picture inside the microchannel to enumerate CD4 cells that have been stained for assay out of the photographed image.

FIG. 3 is a drawing for explaining a depth of field when observing a blood sample in the microchannel.

When an objective lens is brought to a focus on a subject at a certain distance away, a sharp image of the subject as same as one at the focus is also seen within a distance in front (a near point) and behind (a far point) the focus. Here, the distance between the near point and the far point is called a depth of field. When a focus is adjusted within the range of the depth of field, a subject is seen very clearly. Therefore, the depth of field may be referred to as a depth of focus that allows a person to get a sharp image of the subject. In case of photographing inside a microchannel, a plane including the depth of field is photographed.

The depth of field is determined depending on the numerical aperture of an objective lens and the wavelength of light being emitted. Depth of field Z in an (optical) axis direction of light being emitted is calculated by Formula 1 as follows:

Z=λ/(NA)² [Formula 1]

where Z is a depth of field, λ is a wavelength of light being emitted, and NA is a numerical aperture of an objective lens.

For example, suppose that numerical aperture of a 10× objective lens is 0.25 and a green light source with a wavelength of 0.532 μm is used. Then, a depth of field according to Formula 1 will be Z=0.532 μm/(0.25²)=8.512 μm.

Table 1 below sets forth depths of field for objective lenses having different magnifications (numbers in brackets indicate NAs) and different light sources.

TABLE 1
Examples of depth of field

| | 4×(0.1) | 10×(0.3) | 20×(0.4) | 40×(0.5) | 60×(0.6) | 100×(0.95) |
|---|---|---|---|---|---|---|
| BLUE(488 nm) | 46.8μ | 5.42μ | 3.1μ | 1.95μ | 1.36μ | 0.54μ |
| GREEN (532 nm) | 53.2μ | 5.91μ | 3.32μ | 2.13μ | 1.48μ | 0.59μ |
| RED(633 nm) | 63.3μ | 7.03μ | 3.96μ | 2.53μ | 1.76μ | 0.70μ |

Meanwhile, NA of the objective lens is determined by magnification (dimension) of the lens and it is typically indicated together with the magnification. An adequate magnification is selected for the objective lens according to the size of microparticles to be observed. For example, relatively small microparticles can be observed by an objective lens having a relatively high magnification. On the contrary, relatively large microparticles can be observed by an objective lens having a relatively low magnification.

Thus, the depth of field is also influenced by the size of microparticles to be observed.

After the depth of field is obtained from the Formula 1, a microchannel chip is manufactured in such a manner that a depth of the microchannel in the chip is the depth of field obtained.

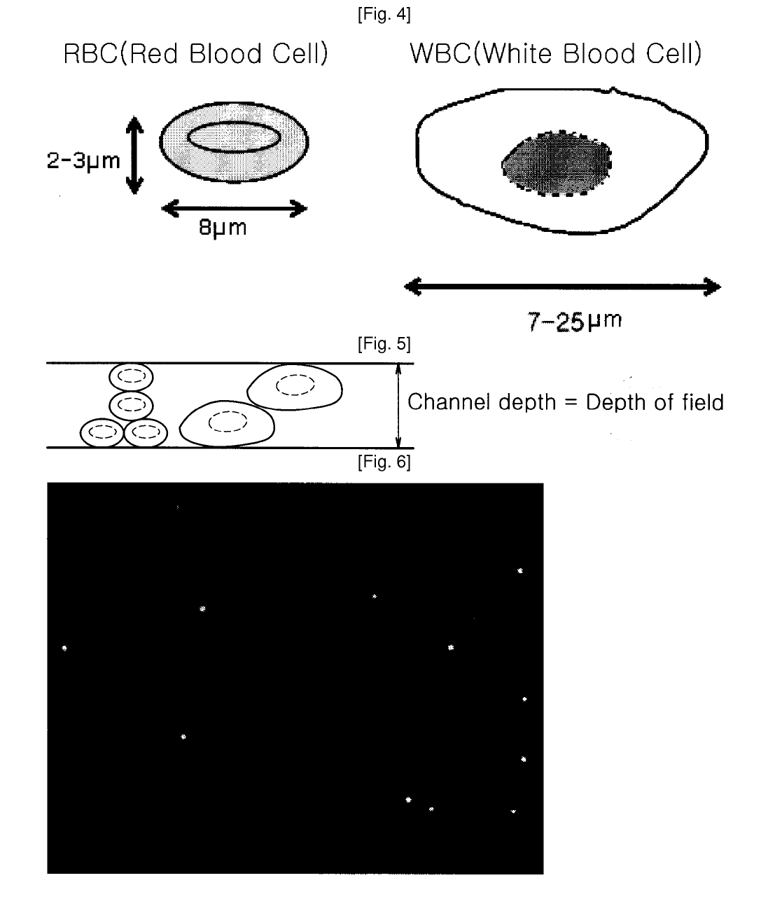

Generally, a white cell in blood is 7-25 μm in size and a red cell in blood is 6-8 μm in size. FIG. 4 compares size of a red blood cell to a white blood cell. For instance, suppose that only CD4 cells in a white blood cell are stained with anti-CD4-PE, which is a staining reagent, and observed by an 4× (NA=0.1) objective lens using a 532 nm wavelength laser as a light source. Then, a depth of field Z=λ/(NA)²=53.2 μm. Thus, if a microchannel chip is manufactured in a manner that its microchannel has a height of about 50 μm, those stained CD4 cells can be observed, not necessarily lysing red blood cells or diluting the blood sample.

As such, when the depth of a microchannel is set prior to the manufacture of a microchannel chip, all CD4 cells are distributed within the depth of field as shown in FIG. 5. Therefore, the blood sample does not need to be diluted to obtain a sharp image of CD4 cells.

Meanwhile, if only CD8 cells, not CD4 cells, in a white blood cell are to be enumerated, CD8 cells are stained with anti-CD8-PE, which is a staining reagent exclusively for CD8 cells.

Moreover, an objective lens with an adequate magnification is selected in consideration of the size of microparticles to be enumerated, and a depth of field is calculated based on the objective lens and wavelength of a light source. In result, a microchannel chip with the calculated depth of field may be manufactured.

To manufacture the microchannel chip, a microchannel pattern is formed on a master by photolithography, and plastic is molded onto the master.

Further, the microchannel chip may be manufactured such that a microchannel therein has a precisely adjusted volume. Thus, by obtaining a total number of microparticles in the microchannel, one can calculate the number of microparticles per unit volume, i.e. the density of microparticles.

The following will now explain how to count or enumerate CD4 cells by using the microchannel chip.

First, 100 μl of human blood is taken. In this embodiment, red blood cells are not separated from white blood cells, nor lysed.

20 μl of mono anti-human CD4 PE, which is a CE4 cell staining reagent, is applied to the blood sample, and incubated at 4° C. for about 30 minutes.

After shaking the mixed liquid several times, the mixture is injected to a microchannel of the microchannel chip. A microparticle counting instrument photographs the microchannel to get an image thereof and counts stained CD4 cells from the image.

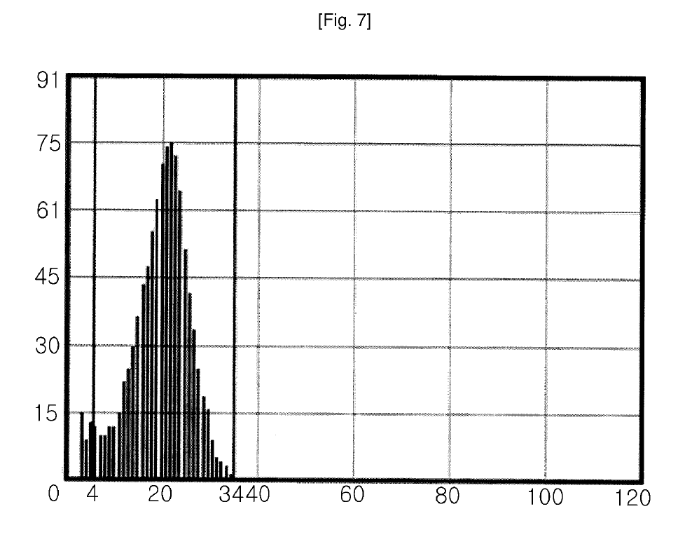

FIG. 6 illustrates a CD4 cell photograph taken by a microparticle counting instrument. Then an image processing is carried out to obtain a graph shown in FIG. 7, which illustrates pixel sizes (x-axis) of a cell image and the number of cells or images (y-axis) with such pixel sizes.

As such, an image of white blood cells bound to the anti-human CD4 PE is obtained by photographing an inside view of the microchannel, and CD4 cells shown on the image are counted. In particular, lines from the origin to the first line (x=4) indicate monocytes counts in the white blood cell, and lines from the first line (x=4) to the next one (x=34) indicate CD4 cell counts.

Table 2 below provides an average density of CD4 cells obtained by repeating the above-described process 10 times.

TABLE 2
Results of test repetition

| | CD4-PE(cells/μ) |
|---|---|
| 1 | 750 |
| 2 | 760 |
| 3 | 820 |
| 4 | 695 |
| 5 | 750 |
| 6 | 760 |
| 7 | 730 |
| 8 | 705 |
| 9 | 730 |
| 10 | 730 |
| Average | 743 |
| SD | 34.7 |
| CV | 4.7 |

As can be seen from the Table 2, deviation for each one of 10 repetition tests is not large.

### INDUSTRIAL APPLICABILITY

Accordingly, it is important that the depth of the microchannel chip of the present invention is made to be a depth of field. In doing so, microparticles are not overlapped or aggregated to give a clear and sharp optical image of microparticles. Further, a microparticle counting instrument can easily count microparticles existing in a microchannel simply by counting microparticles shown on an optical image of the microchannel.

Particularly, the present invention enables to count the number of CD3 cells, CD4 cells or CD8 cells in white blood cells being stained more easily, without lysing or diluting red blood cells. Therefore, the present invention can advantageously used for counting the number of CD3 cells, CD4 cells or CD8 cells in blood of an AIDS patient to monitor the progression of AIDS.

While the present invention has been described with respect to the specific embodiments, it will be apparent to those skilled in the art that various changes and modifications may be made without departing from the spirit and scope of the invention as defined in the following claims.

1. A chip comprising a microchannel in which a sample with microparticles is placed for counting the number of microparticles in the microchannel by optically magnifying and photographing an inside of the microchannel, wherein the depth of the microchannel is same with a depth of field.

2. The chip according to claim 1, wherein the depth of field is determined by Formula 1 as follows:
Z=λ/(NA)² [Formula 1]
wherein Z is a depth of field, λ is a wavelength of light being emitted, and NA is a numerical aperture of an objective lens.

3. The chip according to claim 1, wherein the microparticles are CD3 cells, CD4 cells, or CD8 cells.

4. The chip according to claim 1, wherein the microparticles are white blood cells, bacterium, platelets, or cells.

5. A method for counting CD3, CD4 or CD8 cells in human blood in use of the chip according to claim 1, the method comprising the steps of:
a) applying a staining reagent that selectively binds to the CD3, CD4 or CD8 cells in a blood sample;
b) injecting a mixture prepared in the step a) into the microchannel of the chip; and
c) photographing an inside of the microchannel to obtain an image and count the number of CD3, CD4 or CD8 cells shown in the image.

6. The method according to claim 5, further comprising the step of:
d) calculating a density of the CD3, CD4 or CD8 cells per unit volume of blood, based on the number of the CD3, CD4 or CD8 cells and volume of the microchannel.

7. A method for counting microparticles in a sample by using the chip according to claim 1, the method comprising the steps of:
a) applying a staining reagent that selectively binds to microparticles to be counted in the sample;
b) injecting a mixture prepared in the step a) into the microchannel of the chip; and
c) photographing an inside of the microchannel to obtain an image and count the number of microparticles shown in the image.
